(19) United States
(12) Patent Application Publication
(10) Pub. No.: US 2008/0113386 A1
(43) Pub. Date: May 15, 2008

(54) PREDICTING DIABETIC NEPHROPATHY

(75) Inventors: Ravi Thadhani, Boston, MA (US); S. Ananth Karumanchi, Chestnut Hill, MA (US); Towia Libermann, Chestnut Hill, MA (US)

Correspondence Address: FISH & RICHARDSON PC, P.O. BOX 1022, MINNEAPOLIS, MN, 55440-1022, US

(73) Assignee: The General Hospital Corporation, Boston, MA (US)

(21) Appl. No.: 12/018,945

(22) Filed: Jan. 24, 2008

Related U.S. Application Data
(63) Continuation of application No. 11/595,562, filed on Nov. 9, 2006.
(60) Provisional application No. 60/735,978, filed on Nov. 9, 2005.

(51) Int. Cl. G01N 33/53

(52) U.S. Cl. 435/7.1; 436/86

(57) ABSTRACT

The invention relates to methods and compositions for identifying subjects who are predisposed to having diabetic nephropathy (DN).

### CLAIM OF PRIORITY

This application claims the benefit under 35 USC § 119(e) of U.S. Provisional Patent Application Ser. No. 60/735,978, filed on Nov. 9, 2005, the entire contents of which are hereby incorporated by reference.

### STATEMENT AS TO FEDERALLY SPONSORED RESEARCH

This invention was made with Government support under DK068465 awarded by the National Institute of Diabetes and Digestive and Kidney Diseases of the National Institutes of Health. Thus, the U.S. Government has certain rights in the invention.

### TECHNICAL FIELD

This invention relates to methods of identifying subjects who are at risk for developing Diabetic Nephropathy (DN).

### BACKGROUND

Diabetic nephropathy is kidney disease that develops as a result of diabetes mellitus (DM). DM affects approximately 5% of the U.S. population, and Type 2 Diabetes Mellitus (T2DM) is the most common cause of end stage renal disease (ESRD) in the U.S. Diabetic nephropathy is believed responsible for at least 25% of all renal dialysis patients.

Diabetic nephropathy is thought to be caused by the progressive glycosylation of biomarkers, leading to a progressive loss of renal function. Diabetic nephropathy generally results in a chronic and progressive degradation of kidney function, to the point where the patient must undergo dialysis or receive a transplant to survive. Excretion of low, but abnormal, levels of albumin in the urine is considered a clinical marker of the incipient phase of nephropathy. As the glomeruli become increasingly filled with mesangial matrix products, albuminuria increases and eventually gross proteinuria appears. Microalbuminuria (MA) is defined as excretion of 30 to 300 mg of albumin per day, or an albumin-creatinine ratio between 30 and 300 in a random urine specimen. Clinical proteinuria is defined as excretion of more than 0.5 g of total biomarker a day. However, MA is not a good predictor of ESRD in subjects with T2DM, because not all people who develop MA develop ESRD, and not all subjects who develop ESRD also have evidence of MA.

### SUMMARY

The invention is based, in part, on the discovery that proteomic profiling can be used to identify urine markers that are associated with development of diabetic nephropathy (DN) well before any clinically identifiable alteration in renal function or urine albumin excretion occurs.

The invention provides methods of determining whether a subject is predisposed to develop DN. The methods include generating a subject profile by obtaining a biological sample, e.g., a urine or blood sample, from the subject, measuring the level of at least one biomarker listed in Table 1 or Table 2 (below) in the sample, and comparing the level of the biomarker in the urine sample with a predetermined reference profile. A reference profile can include a profile generated from one or more subjects who are known to be predisposed to develop DN (e.g., subjects in a study who later develop DN), and/or a profile generated from one or more subjects who are not predisposed to develop DN. A “predisposition to develop DN” is a significantly increased risk of developing DN, i.e., the subject is more likely to develop DN than a “normal” subject, i.e., a subject who has diabetes but does not have an increased risk of developing DN. A subject with a predisposition to develop DN is one whose sample has a listed biomarker in amounts that differ from the level of the same biomarker in the reference profile by at least a factor of two, i.e., at least twice or half the level of the biomarker present in the reference profile, where the reference profile represents a subject who is not predisposed to develop DN. Whether an increase or a decrease in a biomarker is associated with a propensity to develop DN is indicated in Table 5.

In some embodiments, the subject has one or more risk factors for developing DN, e.g., duration of diabetes, elevated hemoglobin A1c (HbA1c) levels (e.g., above 8.1%), elevated plasma cholesterol levels, high mean blood pressure, elevated albumin to creatinine ratio (e.g., >0.6), and hyperglycemia (e.g., blood glucose of over 200 mg/dL). In another aspect, the subject does not have microalbuminuria (i.e., excretes less than 30 mg/day) and has normal renal function (i.e., serum creatinine is less than 1.2 mg/dl). In some embodiments, the subject is a Native American, e.g., a Sumi Indian.

In some embodiments, the methods include measuring the level of a plurality of the biomarkers listed in Table 1, e.g., two, three, four, five, six, seven, eight, nine, 10, 11, 12, 13, 14, 15, 16, 17, 18, 19, 20, 21, 22, 23, 24, 25, 26, 27, or all 28 of the biomarkers are measured. In some embodiments, the methods include measuring the level of a plurality of the biomarkers listed in Table 2, e.g., two, three, four, five, six, seven, eight, nine, 10, 11, or all 12 of the biomarkers in Table 2 are measured. The levels of the biomarkers can be used to generate a biomarker profile for the subject.

The methods of the invention can include obtaining a urine sample from a subject, and separating the proteins or protein fragments present into the sample, e.g., by one or more of size, pH, charge, molecular weight, or other physical characteristics. In some embodiments, the methods include treating the sample, e.g., to fragment the proteins and/or improve separation of the proteins or fragments. The separated proteins or fragments can be identified as biomarkers, e.g., by one or more of the characteristics that were used for the separation, e.g., by their molecular weight. In some embodiments, the methods include the use of Matrix Assisted Laser Desorption Ionization Time-of-flight Mass Spectrometry (MALDI-TOF) or Surface-enhanced laser desorption ionization time-of-flight mass spectrometry (SELDI TOF-MS), e.g., as described herein.

In some embodiments, the methods include normalizing for urine creatinine concentrations.

The methods of the invention can include contacting a urine sample obtained from a subject with an array of immobilized biomarker-specific biomolecules and detecting stable or transient binding of the biomolecule to the biomarker, which is indicative of the presence and/or level of a biomarker in the sample. The subject urine biomarker levels can be compared to reference biomarker levels obtained from reference subjects. Reference biomarker levels can further be used to generate a reference profile from one or more reference subjects. In one aspect, the biomarker-specific biomolecules are antibodies, such as monoclonal antibodies. In another aspect, the biomarker-specific biomolecules are antigens, such as viral antigens that specifically recognize the biomarkers. In yet another aspect, the biomarker-specific biomolecules are receptors.

An array of the invention generally includes a substrate having a plurality of addresses, each address having disposed thereon a set of one or more biomolecules, and each biomolecule in the set at a given address specifically detecting the same biomarker; wherein the array includes sufficient addresses to detect at least ten of the biomarkers listed in Table 1, e.g., at least ten of the biomarkers listed in Table 2.

The invention also features a pre-packaged diagnostic kit for detecting a predisposition to DN. The kit can include an array as described above and instructions for using the array to test a urine sample to detect a predisposition to DN. The array can also be used to determine the efficacy of a therapy administered to prevent DN by contacting the array with a urine sample obtained from a subject undergoing a selected therapy. The level of one or more biomarkers in the sample can be determined and compared to the level of the same one or more biomarkers detected in a urine sample obtained from the subject prior to, or subsequent to, the administration of the therapy. Subsequently, a caregiver can be provided with the comparison information for further assessment.

Further, a subject profile can be entered into a computer system that contains, or has access to, a database that includes a plurality of digitally encoded reference profiles (e.g., a computer-readable medium including such databases). Each profile of the plurality has a plurality of values, each value representing a level of a specific biomarker detected in urine of a subject who is predisposed to having DN. In this manner, a single subject profile can be used to identify a subject at risk for developing DN based upon reference values.

The invention also features a computer system for determining whether a subject is predisposed to having DN. The system includes a database that has one or a plurality of digitally-encoded reference profiles, wherein each profile of the plurality has a plurality of values, each value representing a level of a specific biomarker detected in urine of one or more individuals known not to be predisposed to have DN (or known to be so predisposed); and a server including a computer-executable code for causing the computer to: i) receive a profile of a subject including the level of at least one biomarker detected in a urine sample from the subject; ii) identify from the database a matching reference profile that is diagnostically relevant to the subject profile; and iii) generate an indication of whether the subject is predisposed to having DN.

As used herein, the terms “biological molecules” and “biomolecules” are used interchangeably. These terms are meant to be interpreted broadly, and generally encompass polypeptides, peptides, oligosaccharides, polysaccharides, oligopeptides, proteins, oligonucleotides, and polynucleotides. Oligonucleotides and polynucleotides include, for example, DNA and RNA, e.g., in the form of aptamers. Biomolecules also include organic compounds, organometallic compounds, salts of organic and organometallic compounds, saccharides, amino acids, nucleotides, lipids, carbohydrates, drugs, steroids, lectins, vitamins, minerals, metabolites, cofactors, and coenzymes. Biomolecules further include derivatives of the molecules described. For example, derivatives of biomolecules include lipid and glycosylation derivatives of oligopeptides, polypeptides, peptides, and proteins, such as antibodies. Further examples of derivatives of biomolecules include lipid derivatives of oligosaccharides and polysaccharides, e.g., lipopolysaccharides.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a heat map of an array that depicts the correlation between biomarker levels in urine and the risk of developing diabetic nephropathy. Case samples are denoted by N and control samples are denoted by C. Rows represent individual peaks whose intensity values are normalized to [−2,2] as shown in the scale at the bottom. Peak labels are denoted only by the chip on which the peak was detected. In the original figure, red (on the right in the legend) denotes an elevation while green (on the left in the legend) denotes a decrease in expression. CM indicates a biomarker that was identified using a weak cationic exchange chromatography (CM10), protein array; IM indicates a biomarker that was identified using an immobilized metal affinity capture (IMAC30) protein array.

FIG. 2 is a heat map depicting hierarchical clustering of the samples in the training set using the 12-peak signature. Case samples are denoted by N and control samples are denoted by C. Rows represent individual peaks the intensity values of which are normalized to [−2,2] as shown in the scale at the bottom. Peak labels represent the chip on which the peak was detected (IM for IMAC30 and CM for CM10) followed by the molecular weight for the detected peak. In the original figure, red (on the right in the legend) denotes an elevation while green (on the left in the legend) denotes a decrease in expression. The raw data used to produce FIG. 2 is shown below in Table 4.

FIG. 3 is a trace view for one representative peak (CM3807_{—}04) from the 12-peak signature shown in FIG. 2. The detection level for the peak in five case (N) and five control (C) samples in the training set is shown. The level of the peak is elevated in case samples in accordance with FIG. 2.

### DETAILED DESCRIPTION

Diabetic Nephropathy (DN) is associated with significant morbidity and mortality in both the developed and developing world. Currently there are no effective laboratory tests to detect a predisposition to develop DN. Diagnoses are generally made after the subject has developed microalbuminuria, at which time the subject already has some damage to the kidney. The absence of early diagnostic tests has hindered the ability to identify preventive therapeutic agents, which would likely be more successful at preventing development of DN and progression to ESRD.

The present inventors have identified a pattern of urinary proteins that are present 5-10 years before microalbuminuria develops and well before development of albuminuria (a presently accepted clinical hallmark of diabetic nephropathy). This pattern gives a 90% sensitivity in identifying those at risk for the condition several years before the condition actually develops. Proteomic techniques were used to identify these patterns. Thus provided herein are methods of determining whether a subject is predisposed to having DN are provided. In addition, compositions for determining a subject's risk for developing DN are provided.

Signs and Symptoms of Diabetic Nephropathy

Approximately 25% to 40% of patients with DM 1 ultimately develop DN, which progresses through about five predictable stages.

Stage 1 (very early diabetes) is associated with increased demand upon the kidneys, and is indicated by an above-normal glomerular filtration rate (GFR).

In stage 2 (developing diabetes), the GFR remains elevated or has returned to normal, but glomerular damage has progressed to significant microalbuminuria (small but above-normal level of the protein albumin in the urine). Patients in stage 2 excrete more than 30 mg of albumin in the urine over a 24-hour period. Significant microalbuminuria will progress to end-stage renal disease (ESRD). All diabetes patients should be screened for microalbuminuria on a routine (yearly) basis.

In stage 3 (overt, or dipstick-positive diabetes), glomerular damage has progressed to clinical albuminuria. The urine is “dipstick positive,” containing more than 300 mg of albumin in a 24-hour period. Hypertension (high blood pressure) typically develops during stage 3.

In stage 4 (late-stage diabetes), glomerular damage continues, with increasing amounts of protein albumin in the urine. The kidneys' filtering ability has begun to decline steadily, and blood urea nitrogen (BUN) and creatinine (Cr) has begun to increase. The glomerular filtration rate (GFR) decreases about 10% annually. Almost all patients have hypertension at stage 4.

In stage 5 (end-stage renal disease, ESRD), the GFR has fallen to approximately 10 milliliters per minute (<10 mL/min) and renal replacement therapy (e.g., hemodialysis, peritoneal dialysis, or kidney transplantation) is required.

Methods of Identifying At-Risk Subjects

Specific alterations in one or more of the biomarkers listed herein (i.e., in Table 1 or Table 2) are statistically related to the development of DN. These biomarkers serve as early biomarkers for disease, and characterize subjects as at high risk for future disease.

A “subject” profile can also be referred to as a “test” profile. A subject profile can be generated from a sample taken from a subject prior to the development of microalbuminuria, e.g., when the subject is excreting less than 30 mg of albumin a day or has an albumin-creatinine (A/C) ratio of less than 30 in a random urine specimen. Thus, a “subject” profile is generated from a subject being tested for predisposition to DN.

A “reference” profile can also be referred to as a “control” profile. A reference profile can be generated from a sample taken from a normal individual or from an individual known to have a predisposition to DN. The reference profile, or plurality of reference profiles, can be used to establish threshold values for the levels of, for example, specific biomarkers in a sample. A “reference” profile includes a profile generated from one or more subjects having a predisposition to DN or a profile generated from one or more normal subjects.

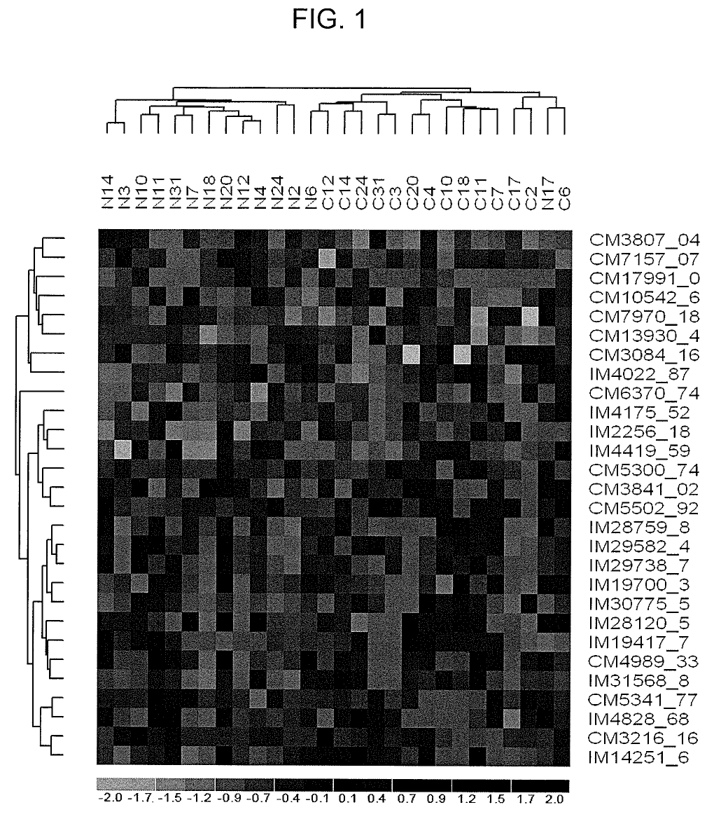

A reference profile can be in the form of an array “signature” or “pattern” of specific identifiable biomarkers. The array signature can be color-coded for easy visual or computer-aided identification. The signature can also be described as a number or series of numbers that correspond to values attributed to the biomarkers identified by the array. The color key shown in FIG. 1 (bottom) provides one example of how values can be attributed to biomarker concentrations identified by an array. “Array analysis,” as used herein, is the process of extrapolating information from an array using statistical calculations such as factor analysis or principle component analysis (PCA).

In addition to being expressed as a signature, a reference profile can be in the form of a threshold value or series of threshold values. For example, a single threshold value can be determined by averaging the values of a series of levels of a single biomarker from subjects having no predisposition to DN. Similarly, a single threshold value can be determined by averaging the values of a series of levels of a single biomarker from subjects having a predisposition to DN. Thus, a threshold value can have a single value or a plurality of values, each value representing a level of a specific biomarker, detected in a urine sample, e.g., of an individual, or multiple individuals, having a predisposition to DN.

As described herein, a subject profile can be used to identify a subject at risk for developing DN based upon a comparison with the appropriate reference profile or profiles. Subjects predisposed to having DN can be identified prior to the development of microalbuminuria by urinalysis. For example, a subject profile that includes the level of at least two biomarkers listed in Table 1 detected in a urine sample from a subject an be compared to a “reference” profile that includes the level of at least two biomarkers detected in a urine sample obtained from a reference subject. If the reference profile is derived from a sample (or samples) obtained from a reference subject having a predisposition to DN, then the similarity of the subject profile to the reference profile is indicative of a predisposition to DN for the tested subject. Alternatively, if the reference profile is derived from a sample (or samples) obtained from a reference subject who does not have a predisposition to DN, then the similarity of the subject profile to the reference profile is not indicative of a predisposition to DN for the tested subject. As used herein a subject profile is “similar” to a reference profile if there is no statistically significant difference between the two profiles. In some embodiments, a subject profile that includes levels that differ by more than a factor of two, e.g., are more than about twice or less than about half, the levels of the same biomarker(s) in the reference profile, when the reference profile is from a reference subject who does not have a predisposition to DN, is indicative of a predisposition to DN in the subject. Whether an increase or a decrease in a biomarker is associated with a propensity to develop DN can be determined from Table 5.

Biomarkers

The methods described herein include the measurement of levels of certain biomarkers, identified herein by molecular weight listed in Table 1. As noted above, the presence of one or more biomarker listed in table 1 or 2 in a subject at a level that is more than about twice, or less than about half, the levels of the same biomarker(s) in the reference profile, when the reference profile is from a reference subject who does not have a predisposition to DN, is indicative of a predisposition to DN in the subject. In some embodiments, e.g., where SELDI-TOF MS is used, reference peak intensity values can be determined using the data in Table 4 or Table 5. Whether an increase or a decrease in a biomarker is associated with a propensity to develop DN can be determined from Table 5.

TABLE 1
28 Biomarkers

| Molecular Weight | Chip/conditions |
|---|---|
| 2256 | IM |
| 3084 | CM |
| 3216 | CM |
| 3807 | CM |
| 3841 | CM |
| 4022 | IM |
| 4175 | IM |
| 4419 | IM |
| 4828 | IM |
| 4989 | CM |
| 5300 | CM |
| 5341 | CM |
| 5502 | CM |
| 6370 | CM |
| 7157 | CM |
| 7970 | CM |
| 10542 | CM |
| 13930 | CM |
| 14251 | IM |
| 17991 | CM |
| 19417 | IM |
| 19700 | IM |
| 28120 | IM |
| 28759 | IM |
| 29582 | IM |
| 29738 | IM |
| 30775 | IM |
| 31568 | IM |

In some embodiments, the methods described herein include the measurement of levels of biomarkers identified herein by molecular weight listed in Table 2. An increase or decrease, as shown in Table 5, in levels of one or more of these biomarkers is indicative of a predisposition to develop DN. Again, in some embodiments, e.g., where SELDI-TOF MS is used, reference peak intensity values can be determined using the data in Table 4 or Table 5, e.g., the data in bold in Table 5. Whether an increase or a decrease in a biomarker is associated with a propensity to develop DN can be determined from Table 5.

TABLE 2
12 Biomarkers

| Molecular Weight | Chip/conditions |
|---|---|
| 3807 | CM |
| 4022 | IM |
| 5502 | CM |
| 7970 | CM |
| 10542 | CM |
| 13930 | CM |
| 17991 | CM |
| 19417 | IM |
| 29582 | IM |
| 29738 | IM |
| 30775 | IM |
| 31568 | IM |

Although the biomarker identified herein are listed by molecular weight only, on eof skill in the art would readily be able to determine their identity. For example, one of skill in the art could readily isolate a polypeptide in a peak identified by SELDI TOF-MS. The polypeptide could then be sequenced using known methods, and the identity of the polypeptide determined. Each peak may include a whole protein, or may include only a portion thereof. Once the identity of the biomarker protein is determined, antibodies can be obtained that bind to the biomarker, e.g., using standard methods. Commercial antibodies can also be used if any are available. Alternatively, antibodies can be generated to the purified polypeptide obtained from the SELDI-TOF MS peak without identifying the protein.

In some embodiments, the peak represents a fragment of a protein, and the presence of the fragment is associated with a predisposition to DN.

Proteomics and Microarrays

the methods described herein use proteomics to predict predisposition to DN well before the development of microalbuminuria. Proteomics is an evolving technology capable of testing for the presence of minute amounts of a vast array of biomarkers using small samples of human tissue. Using proteomic tools, increased or decreased levels of certain biomarkers in a biological sample such as urine, serum, amniotic fluid or placental tissue can be ascertained. The methods described herein can include using urine proteomic analysis as a non-invasive approach to detecting a predisposition to DN. In addition, mathematical algorithms can be used to obtain a complex proteome or “fingerprint.” Such algorithms can include “factor analysis” and “principle component analysis (PCA).” The proteome can consist of a group of biomarkers, some increased in concentration from normal and others decreased, as shown in Table 5, that are diagnostic of a predisposition to DN.

The methods described herein can include the use of an array (i.e., “biochip” or “microarray”) that includes immobilized biomolecules that facilitate the detection of a particular molecule or molecules in a biological sample. Biomolecules that identify the biomarkers described herein can be included in a custom array for detecting subjects predisposed to DN. For example, a custom array can include biomolecules that identify one, two three, five, ten or fifteen or more specific biomarkers listed in Table 1, e.g., all twelve biomarkers in Table 2. Arrays comprising biomolecules that specifically identify selected biomarkers (e.g., as listed in Table 1 or Table 2) can be used to develop a database of information using data provided herein. Additional biomolecules that identify biomarkers that lead to improved cross-validated error rates in multivariate prediction models (e.g., logistic regression, discriminant analysis, or regression tree models) can be included in a custom array of the invention.

Customized arrays provide an opportunity to study the biology of DN. Standard p values of significance (0.05) can be chosen to exclude or include additional specific biomolecules on the microarray that identify particular biomarkers. In addition, the new arrays can be used to determine whether one biomarker alters the strength of association of another biomarker, even if that biomarker itself is not significantly associated with the outcome (e.g., is not by itself predictive of predisposition to DN).

The term “array,” as used herein, generally refers to a predetermined spatial arrangement of binding islands or of biomolecules. Arrays according to the present invention that include biomolecules immobilized on a surface can also be referred to as “biomolecule arrays.” Arrays that comprise surfaces activated, adapted, prepared, or modified to facilitate the binding of biomolecules to the surface can also be referred to as “binding arrays.” Further, the term “array” is used herein to refer to multiple arrays arranged on a surface, such as would be the case where a surface bears multiple copies of an array. Such surfaces bearing multiple arrays may also be referred to as “multiple arrays” or “repeating arrays.” The use of the term “array” herein encompasses biomolecule arrays, binding arrays, multiple arrays, and any combination thereof; and the appropriate meaning will be apparent from context. An array can include biomarker-specific biomolecules that detect biomarkers altered in a subject who has a predisposition to DN.

The biological samples used in the new methods and with the new arrays include fluid or solid samples from any tissue of the body including excretory fluids such as urine. Non-urine samples include, but are not limited to serum and plasma.

An array of the invention comprises a substrate. By “substrate” or “solid support” or other grammatical equivalents, is meant any material appropriate for the attachment of biomolecules and is amenable to at least one detection method. There are many possible substrates including, but not limited to, glass and modified or functionalized glass, plastics (including acrylics, polystyrene and copolymers of styrene and other materials, polypropylene, polyethylene, polybutylene, polyurethanes, and TEFLON®), polysaccharides, nylon or nitrocellulose, resins, silica or silica-based materials including silicon and modified silicon, carbon, metals, inorganic glasses, plastics, ceramics, and a variety of other polymers. In addition, as is known in the art, the substrate may be coated with any number of materials, including polymers, such as dextrans, acrylamides, gelatins or agarose. Such coatings can facilitate the use of the array with a biological sample derived from urine or serum.

A “planar” array will generally contain addressable locations (e.g., “pads”, “addresses” or “micro-locations”) of biomolecules in an array format. The size of the array will depend on the composition and end use of the array. Arrays containing from about two to many thousands different biomolecules, or sets of biomolecules (e.g., redundant sets), can be made. Generally, the array will comprise from two to as many as 100,000 or more, e.g., 5, 10, 25, 50, 100, or more different biomolecules, depending on the end use of the array. A microarray of the invention will generally comprise at least one biomolecule that identifies or “captures” a biomarker, such as a protein or polypeptide, present in a biological sample. In some embodiments, the compositions described herein may not be in an array format; that is, for some embodiments, compositions comprising a single biomolecule may be made as well. In addition, in some arrays, multiple substrates may be used, either of different or identical compositions. Thus, for example, large planar arrays may comprise a plurality of smaller substrates.

As an alternative to planar arrays, bead-based assays in combination with flow cytometry have been developed to perform multi-parametric immunoassays. In bead-based assay systems the biomolecules can be immobilized on addressable microspheres. Each biomolecule for each individual immunoassay is coupled to a distinct type of microsphere (i.e., “microbead”) and the immunoassay reaction takes place on the surface of the microspheres. Dyed microspheres with discrete fluorescence intensities are loaded separately with their appropriate biomolecules. The different bead sets carrying different capture probes can be pooled as necessary to generate custom bead arrays. Bead arrays are then incubated with the sample in a single reaction vessel to perform the immunoassay.

Product formation of the biomarker with their immobilized capture biomolecules can be detected with a fluorescence-based reporter system. Biomarkers can either be labeled directly by a fluorogen or detected by a second fluorescently labeled capture biomolecule. The signal intensities derived from captured biomarkers are measured in a flow cytometer. The flow cytometer first identifies each microsphere by its individual color code. Second, the amount of captured biomarkers on each individual bead is measured by the second color fluorescence specific for the bound target. This allows multiplexed quantitation of multiple targets from a single sample within the same experiment. Sensitivity, reliability and accuracy are compared to standard microtiter ELISA procedures. With bead-based immunoassay systems biomarkers can be simultaneously quantified from biological samples. An advantage of bead-based systems is the individual coupling of the capture biomolecule to distinct microspheres.

Thus, microbead array technology can be used to sort biomarkers bound to a specific biomolecule using a plurality of microbeads, each of which can carry about 100,000 identical molecules of a specific anti-tag biomolecule on the surface of a microbead. Once captured, the biomarker can be handled as fluid, referred to herein as a “fluid microarray.”

Microarrays as described herein can be biochips that include a high density of immobilized arrays of recognition molecules (e.g., antibodies), where biomarker binding is monitored indirectly (e.g., via fluorescence). In addition, an array can be of a format that involves the capture of biomarkers by biochemical or intermolecular interaction, coupled with direct detection using a label-free detection method. Such methods include, but are not limited to, surface plasmon resonance, micro-electro-mechanical systems (e.g., cantilevers), semiconductor nanowires, and mass spectrometry (MS).

Arrays and microarrays that can be used with the new methods to detect the biomarkers described herein can be made, for example, according to the methods described in U.S. Pat. Nos. 6,329,209; 6,365,418; 6,406,921; 6,475,808; and 6,475,809, and U.S. patent application Ser. No. 10/884,269, which are incorporated herein by reference in their entirety. New arrays to detect specific sets of biomarkers described herein can also be made using the methods described in these patents.

Arrays and microarrays described herein further include arrays that have pathogen-encoded biomarker-binding proteins immobilized on a solid surface. For example, poxvirus genes encoding binding activities for TNF type I and type II interferons, interleukin (IL)-1 beta, IL-18, and beta-chemokines have been identified. These high-affinity receptors have the potential to act as surrogate antibodies in a number of applications in biomarker quantification and purification and could be potentially useful reagents to complement the existing panel of anti-biomarker, monoclonal, polyclonal, or engineered antibodies that are currently available.

In many embodiments, immobilized biomolecules, or biomolecules to be immobilized, are proteins. One or more types of proteins may be immobilized on a surface. In certain embodiments, the proteins are immobilized using methods and materials that minimize the denaturing of the proteins, that minimize alterations in the activity of the proteins, or that minimize interactions between the protein and the surface on which they are immobilized.

Surfaces for immobilization of biomolecules may be of any desired shape (form) and size. Non-limiting examples of surfaces include chips, continuous surfaces, curved surfaces, flexible surfaces, films, plates, sheets, tubes, and the like. Surfaces preferably have areas ranging from approximately a square micron to approximately 500 cm². The area, length, and width of surfaces according to the present invention may be varied according to the requirements of the assay to be performed. Considerations may include, for example, ease of handling, limitations of the material(s) of which the surface is formed, requirements of detection systems, requirements of deposition systems (e.g., arrayers), and the like.

In certain embodiments, it is desirable to employ a physical means for separating groups or arrays of binding islands or immobilized biomolecules: such physical separation facilitates exposure of different groups or arrays to different solutions of interest. Therefore, in certain embodiments, arrays are situated within wells of 96, 384, 1536, or 3456 microwell plates. In such embodiments, the bottoms of the wells may serve as surfaces for the formation of arrays, or arrays may be formed on other surfaces and then placed into wells. In certain embodiments, such as where a surface without wells is used, binding islands may be formed or biomolecules may be immobilized on a surface and a gasket having holes spatially arranged so that they correspond to the islands or biomolecules may be placed on the surface. Such a gasket is preferably liquid tight. A gasket may be placed onto a surface at any time during the process of making the array and may be removed if separation of groups or arrays is no longer necessary.

The immobilized biomolecules can bind to molecules present in a biological sample overlying the immobilized biomolecules. Alternatively, the immobilized biomolecules modify or are modified by molecules present in a biological sample overlying the immobilized biomolecules. For example, a biomarker present in a biological sample can contact an immobilized biomolecule and bind to it, thereby facilitating detection of the biomarker. Alternatively, the can contact a biomolecule immobilized on a solid surface in a transient fashion and initiate a reaction that results in the detection of the biomarker absent the stable binding of the biomarker to the biomolecule.

Modifications or binding of biomolecules in solution or immobilized on an array may be detected using detection techniques known in the art. Examples of such techniques include immunological techniques such as competitive binding assays and sandwich assays; fluorescence detection using instruments such as confocal scanners, confocal microscopes, or CCD-based systems and techniques such as fluorescence, fluorescence polarization (FP), fluorescence resonant energy transfer (FRET), total internal reflection fluorescence (TIRF), fluorescence correlation spectroscopy (FCS); colorimetric/spectrometric techniques; surface plasmon resonance, by which changes in mass of materials adsorbed at surfaces may be measured; techniques using radioisotopes, including conventional radioisotope binding and scintillation proximity assays so (SPA); mass spectroscopy, such as matrix-assisted laser desorption/ionization mass spectroscopy (MALDI) and MALDI-time of flight (TOF) mass spectroscopy; ellipsometry, which is an optical method of measuring thickness of protein films; quartz crystal microbalance (QCM), a very sensitive method for measuring mass of materials adsorbing to surfaces; scanning probe microscopies, such as atomic force microscopy (AFM) and scanning electron microscopy (SEM); and techniques such as electrochemical, impedance, acoustic, microwave, and IR/Raman detection. See, e.g., Mere et al., Drug Discov. Today 4(8):363-369 (1999), and references cited therein; Lakowicz, Principles of Fluorescence Spectroscopy, 2nd Edition, Plenum Press (1999).

Arrays suitable for identifying a subject who has a propensity to develop DN can be included in kits. Such kits may also include, as non-limiting examples, reagents useful for preparing biomolecules for immobilization onto binding islands or areas of an array, reagents useful for detecting modifications to immobilized biomolecules, reagents useful for detecting binding of biomolecules from solutions of interest to immobilized biomolecules, and/or instructions for use. Likewise, arrays comprising immobilized biomolecules may be included in kits. Such kits may also include, as non-limiting examples, reagents useful for detecting modifications to immobilized biomolecules or for detecting binding of biomolecules from solutions of interest to immobilized biomolecules.

Theranostics

The invention provides compositions and methods for the identification of subjects who are at high risk for DN such that a theranostic approach can be taken to test such individuals to determine the effectiveness of a particular therapeutic intervention (pharmaceutical or non-pharmaceutical) and to alter the intervention to 1) reduce the risk of developing adverse outcomes and 2) enhance the effectiveness of the intervention. Thus, in addition to diagnosing or confirming the predisposition to DN, the methods and compositions described herein also provide a means of optimizing the treatment of a subject having such a disorder. The invention provides a theranostic approach to treating and preventing DN by integrating diagnostics and therapeutics to improve the real-time treatment of a subject. Practically, this means creating tests that can identify which patients are most suited to a particular therapy, and providing feedback on how well a drug is working to optimize treatment regimens. The markers provided herein are particularly adaptable for use in diagnosis and treatment because they are available in easily obtained body fluids such as urine.

Within the clinical trial setting, a theranostic method or composition of the invention can provide key information to optimize trial design, monitor efficacy, and enhance drug safety. For instance, “trial design” theranostics can be used for patient stratification, determination of patient eligibility (inclusion/exclusion), creation of homogeneous treatment groups, and selection of patient samples that are representative of the general population. Such theranostic tests can therefore provide the means for patient efficacy enrichment, thereby minimizing the number of individuals needed for trial recruitment. “Efficacy” theranostics are useful for monitoring therapy and assessing efficacy criteria. Finally, “safety” theranostics can be used to prevent adverse drug reactions or avoid medication error.

Statistical Analyses

The data presented herein can be used to create a database of information related to predisposition to DN. Classification and prediction provide a statistical approach to interpreting and utilizing the data generated by an array as shown in FIG. 1. Prediction rules can be selected based on cross-validation, and further validating the chosen rule on a separate cohort. A variety of approaches can be used to generate data predictive of a predisposition to DN based on biomarker levels as provided herein, including discriminant analysis, logistic regression, and regression trees.

Discriminant analysis attempts to find a plane in the multivariate space of the marker data such that, to the extent possible, cases appear on one side of this plane, and controls on the other. The coefficients that determine this plane constitute a classification rule: a linear function of the marker values, which is compared with a threshold. In Bayesian classification, information on the probability of a subject being predisposed to having DN that is known before the data are obtained can be employed. For example the prior probability of being a case can be set to about 0.5; for a screening test applied to a general population the corresponding probability will be approximately 0.05. A subject is classified as having a predisposition to DN if the corresponding posterior probability (i.e., the prior probability updated using the data) exceeds 0.5.

Additional patient information can be combined with the data provided using a method described herein. These data can be combined in a database that analyzes the information to identify trends that complement the present biomarker data. Results can be stored in an electronic format.

The present methods use biomarker levels for determining the risk for developing DN. The methods provided herein can be combined with the patient history to enhance the reliability of the prediction. Thus, information concerning the patient can be considered in conjunction with the results of the methods described herein to enhance reliability. Such information includes, but is not limited to, age, weight or body mass index, duration of diabetes, hemoglobin A1C levels, serum creatinine levels, urine albumin creatinine ratio, gender, blood pressure, genetic history, and other such parameters or variables.

Confounders and covariates in the analysis of data generated to establish guidelines for predisposition to DN can be included in the database of information.

Additional analyses can be performed to identify subjects at risk for DN. Such analyses include bivariate analysis of each of the primary exposures, multivariate models including variables with a strong relationship (biologic and statistical) with outcomes, methods to account for multiple critical exposures including variable reduction using factor analysis, and prediction models.

For bivariate analysis, the mean level of each primary exposure between cases and controls using a 2-sample t-test or Wilcoxon Rank Sum test, as appropriate, can be conducted. If the association appears linear, a trend can be analyzed using the Mantel Haenszel test. Data can be assembled into less fine categories (e.g., tertiles) using the distribution of the controls, and one can examine these as indicator variables in multivariable analysis.

For multivariate analyses, data can be correlated between two control groups, one matched and another not matched. In both matched and unmatched analyses, the independent effects of all primary exposures of interest can be examined using logistic regression (with conditional models in matched analyses) models. The models can include a minimum number of covariates to test the main effect of specific predictors. The effect of specific biomarkers can be determined in addition to development of DN after accounting for confounders or potentially mediating variables.

Logistic regression models take the general form [ln(pᵢ/1−pᵢ)=b₀+b₁X1ᵢ+b₂X2ᵢ+ . . . +bₙXₙᵢ+e], where pᵢ is the probability of DN, b₀ represents the intercept of the fitted line, b₁ is the coefficient associated with a unit increase in the level of a specific biomarker, b₂ . . . bₙ are the coefficients associated with confounding covariates X2 . . . Xn, and e is an error term. The odds ratio associated with a unit increase in the level of a specific biomarker is estimated by exponentiating the coefficient b₁, and the 95% confidence interval surrounding this point estimate is estimated by exponentiating the term (b₁±1.96 (standard error of b₁)). In models with more than one bₙ covariate, the effect of b₁ can be interpreted as the effect of the specific biomarker level on risk of DN after adjustment for levels of confounding covariates included in the model.

In factor analysis, specific biomarkers can be reduced to a smaller number of inter-correlated biomarkers. Factor scores derived from rotated principal components (which are normally distributed continuous variables) can be modeled instead of original biomarker levels in regression analyses predicting predisposition to develop DN. This model-building strategy is similar to that described above, but modeling factor scores allowing the identification of specific biomarker signatures as predictive of outcomes independently of other biomarker signatures, or independently of important pre-specified confounding or mediating variables.

The diverse array of potentially inter-correlated biomarkers or other biomolecules derived from array experiments can be reduced with factor analysis using principal component analysis. Principal component analysis identifies subsets of correlated variables that group together. These subsets define components: mathematically derived variables that are uncorrelated with each other and that explain the majority of the variance in the original data. Principal components analysis (PCA) attempts to identify a minimum number of components needed to effect a diagnosis. After identification, components are transformed, or rotated, into interpretable factors. Interpretation is based on the pattern of correlations between the factors and the original independent variables; these correlations are called loadings. In array experiments, factor patterns represent domains or distinct groupings of biomarker or other biomolecules underlying the overall relationships among the original array of putatively independent biomarker levels. These groupings may be considered as biomolecule, e.g., biomarker, signature patterns.

Variables can be transformed to improve normality, although principal components are fairly robust to normality deviations. Variables included in the factor analysis include all biomarker levels included in an array experiment, for example. In most cases the minimum number of components are selected based on components whose eigenvalues exceed unity. Eigenvalues are the sum of the squared correlations between the original independent variables and the principal components and represent the amounts of variance attributable to the components.

To avoid over-factored models one generally excludes components with eigenvalues equal to or barely exceeding unity that lie below the inflection point on a screen plot and that do not contribute additional clarity to the resultant factor pattern. To produce interpretable factors, the minimum number of principal components can be rotated using the orthogonal varimax method. This orthogonal rotation is a transformation of the original components that produces factors uncorrelated with each other (representing unique independent domains), but highly correlated with unique subsets of the original biomarkers. In general, loadings (correlations between the factors and the original independent variables; range −1.0 to 1.0) greater than about 0.30 are used to interpret the resulting factor pattern. Similarities between loadings on the same factor within selected subgroups (for example, Asian versus White women) can be evaluated using coefficients of congruence. The coefficient of congruence approaches unity when factor loadings are identical between subgroups.

Although factor analysis is not a strict hypothesis testing methodology, one can use Bartlett's method, which gives a value distributed approximately as chi-square, to test the null hypothesis that the first dominant factor may be significant, but remaining factors explain only error variance and are not significant. Confirmatory factor analyses can be conducted to assess whether an empirically determined model (e.g., a three factor solution with two independent variables loading on two factors) provides a better fit to the data than a model with all independent variables loading on a single factor (the null hypothesis model). Three goodness-of fit indices are generally employed: (i) the maximum likelihood goodness-of-fit index, which gives a value distributed as chi-square and where a smaller value indicates a better fit to the data, (ii) Bentler's non-normed fit, and (iii) Bentler and Bonett's comparative fit indices, where higher values (range, 0 to 1.0) indicate a better fit.

Databases and Computerized Methods of Analyzing Data

A database generated from the methods provided herein and the analyses described above can be included in, or associated with, a computer system for determining whether a subject is predisposed to having DN. The database can include a plurality of digitally encoded “reference” (or “control”) profiles. Each reference profile of the plurality can have a plurality of values, each value representing a level of a specific biomarker detected in urine of a individual predisposed to having DN. Alternatively, a reference profile can be derived from a individual that is normal. Both types of profiles can be included in the database for consecutive or simultaneous comparison to a subject profile. The computer system can include a server containing a computer-executable code for receiving a profile of a subject and identifying from the database a matching reference profile that is diagnostically relevant to the subject profile. The identified profile can be supplied to a caregiver for diagnosis or further analysis.

Using standard programs, electronic medical records (EMR) can be accumulated to provide a database that combines biomarker antagonist data with additional information such as the BMI of a patient or any other parameters useful for predicting the risk of developing DN. Patient information can be randomly assigned a numerical identifier to maintain anonymity with testing laboratories and for security purposes. All data are can be stored on a network that provides access to multiple users from various geographic locations.

Thus, the various techniques, methods, and aspects of the invention described above can be implemented in part or in whole using computer-based systems and methods. Additionally, computer-based systems and methods can be used to augment or enhance the functionality described above, increase the speed at which the functions can be performed, and provide additional features and aspects as a part of or in addition to those of the invention described elsewhere in this document. Various computer-based systems, methods, and implementations in accordance with the above-described technology are presented below.

A processor-based system can include a main memory, preferably random access memory (RAM), and can also include a secondary memory. The secondary memory can include, for example, a hard disk drive and/or a storage drive (e.g., a removable storage drive), representing a floppy disk drive, a magnetic tape drive, an optical disk drive, etc. The storage drive reads from and/or writes to a machine-readable (computer-readable) storage medium, which refers to a floppy disk, magnetic tape, optical disk, and the like, which is read by and written to by a storage drive. As will be appreciated, the machine-readable storage medium can comprise computer software and/or data, e.g., in the form of tables, databases, or spreadsheets.

In alternative embodiments, the secondary memory may include other similar means for allowing computer programs or other instructions to be loaded into a computer system. Such means can include, for example, a storage unit and an interface. Examples of such can include a program cartridge and cartridge interface, a movable memory chip (such as an EPROM or PROM) and associated socket, and other storage units (e.g., removable storage units) and interfaces, which allow software and data to be transferred from the storage unit to the computer system.

Computer programs (also called computer control logic) are stored in main memory and/or secondary memory. Computer programs can also be received via a communications interface. Such computer programs, when executed, enable the computer system to perform the methods described herein. In particular, the computer programs, when executed, enable the processor to perform the features or steps of the new methods. Accordingly, such computer programs represent controllers of the computer system.

In an embodiment where the elements are implemented using software, the software may be stored in, or transmitted via, a computer-readable medium and loaded into a computer system using a removable storage drive, hard drive or communications interface. The control logic (software), when executed by the processor, causes the processor to perform the functions of the methods described herein.

In another embodiment, the elements are implemented primarily in hardware using, for example, hardware components such as Programmable Array Logic devices (PALs), application specific integrated circuits (ASICs), or other hardware components. Implementation of a hardware state machine so as to perform the functions described herein will be apparent to persons skilled in the relevant art(s). In yet another embodiment, elements are implemented using a combination of both hardware and software.

In another embodiment, the computer-based methods can be accessed or implemented over the World Wide Web by providing access via a Web Page to the systems and databases described herein. The Web Page can be identified by a Universal Resource Locator (URL). The URL denotes both the server machine and the particular file or page on that machine. In this embodiment, it is envisioned that a consumer or client computer system interacts with a browser to select a particular URL, which in turn causes the browser to send a request for that URL or page to the server identified in the URL. Typically the server responds to the request by retrieving the requested page and transmitting the data for that page back to the requesting client computer system (the client/server interaction is typically performed in accordance with the hypertext transport protocol (“HTTP”)). The selected page is then displayed to the user on the client's display screen. The client may then cause the server containing a computer program of the invention to launch an application to, for example, perform an analysis according to the invention.

### EXAMPLES

The invention is further described in the following examples, which serve to illustrate, but not to limit the scope of the invention described in the claims.

### Example 1

This Example describes the identification of biomarkers that are statistically related to the predisposition of a human subject to develop diabetic nephropathy (DN). Although the data presented herein were obtained from a population of Native Americans, it is reasonable to believe that these results are applicable to subjects of other ethnic and racial backgrounds.

Methods

The Experiments described herein were performed as part of a nested case-control study of Pima Indians with Type 2 Diabetes Mellitus (T2DM). All cases and controls came from a cohort of Pima Indians and the closely related Tohono O'odham (Papago) Indians, who live in the Gila River Indian Community in central Arizona and participate in a comprehensive longitudinal diabetes study (Bennett et al., Lancet. 2:125-128 (1971)). Since 1965, each member of the population five years old and older is invited to have a research examination approximately every two years. These examinations include measurements of venous plasma glucose, obtained 2 hours after a 75 g oral glucose load, and an assessment of various complications of diabetes. Diabetes is diagnosed by World Health Organization criteria (Diabetes mellitus. Report of a WHO Study Group. World Health Organ Tech Rep Ser. 1985; 727:1-113) and the date of diagnosis is determined from these research examinations or from review of clinical records if diabetes is diagnosed between research examinations in the course of routine medical care. A urine specimen is collected at each examination and is assayed for albumin concentration with a nephelometric immunoassay using a monospecific antiserum to human albumin (Vasquez et al., Diabetologia. 26:127-33 (1984)) and for creatinine concentration using a modification of the Jaffe method (Chasson et al., Tech Bull Regist Med Techn. 30:207-212 (1960)). Albumin excretion is expressed as the ratio of urinary albumin to urinary creatinine (mg/g) from a single untimed urine specimen.

The baseline and follow-up characteristics of the subjects are illustrated in

TABLE 3

| | | |
|---|---|---|
| TABLE 3 (* P ≦ 0.05) | Cases (n = 31) | Controls (n = 31) |

Baseline Characteristics

| | | |
|---|---|---|
| Age (years) | 36 ± 10 | 37 ± 8 |
| Gender (% Female) | 80 | 80 |
| Systolic Blood Pressure (mm Hg) | 120 ± 16 | 121 ± 19 |
| Diastolic Blood Pressure (mm Hg) | 74 ± 12 | 74 ± 11 |
| Serum Creatinine (mg/dl) | 0.66 ± 14 | 0.71 ± 11 |
| Hemoglobin A1C (%) | 9.9 ± 2.5 | 8.0 ± 2.7 |
| Urine Albumin/Creatinine (A/C) | 14 ± 9 | 9 ± 6 |
| Ratio (mg/g) | | |

Follow Up Characteristics

| | | |
|---|---|---|
| Age (years) | 52 ± 9 | 51 ± 9 |
| Systolic Blood Pressure (mm Hg) | 138 ± 19* | 124 ± 20 |
| Diastolic Blood Pressure (mm Hg) | 78 ± 10* | 73 ± 11 |
| Serum Creatinine (mg/dl) | 0.80 ± 0.32 | 0.75 ± 12 |
| Hemoglobin A1C (%) | 10.3* | 9.0 |
| Urine Albumin Creatinine Ratio | 1504 ± 1936* | 16 ± 7 |
| (mg/g) | | |

* P value < 0.05

Urine samples were collected at baseline and 10 years later in 31 cases and 31 contemporaneous controls that were matched for age (±5 years), gender, duration of diabetes (±5 years), and body mass index (±5 kg/m²). The two populations were defined as follows: Cases (n=31) were normoalbuminuric (ACR<30 mg/g) and had a normal serum creatinine concentration (≦1.2 mg/dl) at baseline, but developed DN (A/C>300 mg/g) within 10 years. Controls (n=31) were also normoalbuminuric and had a normal serum creatinine concentration (≦1.2 mg/dl) at baseline, and did not progress to microalbuminuria within 10 years of follow-up, i.e., remained normoalbuminuric after 10 years.

Proteomic profiling using surface-enhanced laser desorption/ionization time-of-flight mass spectrometry (SELDI TOF-MS, Ciphergen, Fremont, Calif.) was performed on baseline urine samples collected and stored at −80° C. SELDI TOF-MS was carried out in duplicate on Ciphergen PROTEINCHIP™ arrays using an optimized fully automated protocol on the liquid-handling robot (Biomek FX™, Beckman Coulter) as described previously (Aivado et al., Clin Chem Lab Med. 43:133-40 (2005)).

Weak cationic exchange chromatography (CM10, Ciphergen) and immobilized metal affinity capture (IMAC30, Ciphergen) protein arrays were used for profiling.

Weak cationic exchange chromatography protein arrays (CM10 PROTEINCHIP™ arrays; Ciphergen) were pretreated with 10 mM HCl for 5 minutes, and then rinsed with HPLC grade water. Subsequently, the arrays were loaded onto a 192-well bioprocessor, and equilibrated with 20 mM ammonium acetate/0.1% Triton X-100 (Sigma), pH 6.0. Ten μl cell lysate and 50 μl 20 mM ammonium acetate/0.1% Triton X-100 were dispensed onto each array spot, and incubated for one hour. The incubation comprised 60 cycles of pipetting the sample mixture up and down for 30 seconds. Array spots were washed 3×5 minutes with 75 μl 20 mM ammonium acetate/0.1% Triton X-100 and 1×5 minutes with 75 μl water.

Immobilized metal affinity capture arrays (IMAC30 PROTEINCHIP™ arrays; Ciphergen, Fremont, Calif.) were incubated with 100 mM CuSO₄ for 25 minutes and loaded onto a 192-well bioprocessor. Subsequently, the arrays were equilibrated with 50 mM NaCl, 100 mM NaH₂PO₄, pH 7.0. Ten μl cell lysate and 40 μl 50 mM NaCl, 100 mM NaH₂PO₄, pH 7.0 were dispensed onto each array spot and incubated for an hour. Array spots were washed 3×5 minutes with 75 μl 500 mM NaCl, 100 mM NaH₂PO₄, pH 7.0 to remove non-specifically bound proteins and then washed 5 minutes with 75 μl water.

SPA (sinapinic acid; Fluka), the matrix molecule, was prepared as a saturated solution in 50% acetonitrile/0.5% trifluoroacetic acid, and then diluted 1:1 in 50% acetonitrile/0.5% trifluoroacetic acid. After air drying arrays, 2×1 μl and 2×0.75 μl of SPA were dispensed to each spot of the hydrophobic, cationic exchange and IMAC arrays respectively, again using the BIOMEK FX™ Laboratory Automation Workstation (Beckman Coulter, Fullerton, Calif.) equipped with a 96-channel 200 μl head. The arrays were air-dried again, and immediately analyzed.

Individual protein peaks, which represent polypeptides of the same or similar molecular weight, were detected using the Ciphergen BIOMARKER WIZARD™ mass spectra analysis software (Ciphergen, Fremont, Calif.). To identify distinct and significant peaks, a signal-to-noise ratio cut-off of 2 (Aivado et al., Clin. Chem. Lab. Med. 43:133-40 (2005)), which selects only peaks whose signal level is significantly above the calculated background noise, was used. The urine samples were interrogated for the full range of protein peaks whose molecular mass lies between 2,000 Da-40,000 Da. The urine protein peak data were normalized using the total ion current method as described previously (Aivado et al., (2005) supra). Following manufacturer's specifications, the normalization step was corrected for the baseline by excluding noise from the matrix molecule between 0 and 2,000 Da. The intra-assay coefficient of variation was approximately 20%, which is within an acceptable range for SELDI studies Cancer Res. (Aivado et al., (2005) supra; Rogers et al., 63:6971-83 (2003)). All analyses were conducted with and without normalization for urine creatinine concentrations.

A predictive peak signature was defined as a subset of measured peaks that can be used to predict whether a sample indicates that a subject will develop DN or not based on the urine sample's baseline protein profile for the peaks in the signature. To identify a predictive peak signature that can be tested on an independent set for its accuracy, the subjects were randomly divided into a training set and a validation set. The training set was used to ascertain the predictive signature, which was then applied to the independent validation set that had not been used in the initial identification of the predictive signature. The training set consisted of 14 cases and matched controls, and the validation set consisted of 17 cases and matched controls. The average time elapsed from documentation of normoalbuminuria to evidence of overt nephropathy for the cases in the training and validation sets were similar (119.64±7.29 months vs. 120.88±5.82 months, p>0.05).

A set of descriptive peaks was identified on the training data set using t-tests and a threshold of p<0.05. The descriptive peaks were refined using the accuracy of its subsets as predictor peaks on the training set. The best performing (highest leave-one-out accuracy) subset of the descriptive peaks was chosen as the predictive profile and was subsequently applied on the validation set. Class prediction was done using the weighted voting algorithm where the descriptive peaks were used to perform leave-one-out cross validation (Golub et al., Science. 286:531-7 (1999)). This procedure was initiated on the training set, a sample was left out, and a predictor set of peaks that distinguished the two groups was built and used to predict the class of the sample left-out. This procedure was cycled through all samples individually. The accuracy of the predictor equaled the total number of correctly predicted left-out sample. The p-value for the predictor accuracy was calculated using Fisher's exact test. Multivariate analysis to control for confounding was carried out by binary logistic regression with categorical or continuous covariates as appropriate.

A hierarchical clustering technique was used to construct an Unweighted Pair Group Method with Arithmetic-mean (UPGMA) tree using Pearson's correlation as the metric of similarity (Sneath and Sokal, Nature. 193:855-60 (1962)). This tree represents the similarity between samples based on the proteome profile observed on the chips for the predictive peak set.

Results

The baseline and follow-up characteristics are shown in Table 3. At baseline, the two groups were similar with respect to most characteristics (age, gender, blood pressure, serum creatinine, urine albumin-to-creatinine ratios) except for HbA1C levels, which tended to be higher in cases. Furthermore, at baseline 5 controls and 4 cases were taking some form of anti-hypertensive medication, and 8 controls and 4 cases had documented evidence of retinopathy. At follow-up, cases had significantly higher blood pressures, HbA1C levels, and as expected, urine albumin-to-creatinine measures.

SELDI-TOF MS detected 714 individual protein peaks (337 on CM10 and 377 on IMAC30) in urine samples, which represent polypeptides of the same or similar molecular weight. The intensity for each of the 714 peaks on all 62 samples was calculated by the Ciphergen BIOMARKER WIZARD™ software. Using the training set (14 case vs. 14 control) 28 descriptive peaks were identified that differentiated well between the two groups (t-test p<0.05). The 28 peaks are listed by molecular weight in Table 1, above. The results are shown in FIG. 1, which shows the hierarchical clustering of the samples in the training set using the 28-peak signature. Case samples are denoted by N and control samples are denoted by C. Rows represent individual peaks (molecular weights shown) the intensity values of which are normalized to [−2,2] as shown in the scale at the bottom. Peak labels are denoted only by the chip on which the peak was detected. Red denotes an elevation while green denotes a decrease in expression.

The intensity values for the 28 peaks shown in Table 1 and FIG. 1 are shown in Table 4.

TABLE 4
Raw data for FIG. 1, (case samples)

| peak | N18 | N12 | N14 | N24 | N17 | N6 | N10 |
|---|---|---|---|---|---|---|---|
| CM10542_6 | 0.708114 | 0.672843 | 0.129853 | 0.210309 | 0.337867 | −0.01833 | 0.060905 |
| CM13930_4 | −0.02838 | 0.216755 | 0.053921 | 0.065553 | 0.301598 | 0.135915 | 0.047764 |
| CM17991_0 | 0.510934 | 0.262465 | −0.01948 | 0.009226 | −0.01052 | 1.001472 | 0.36672 |
| CM3084_16 | −0.23427 | 0.226246 | 0.447604 | 0.450791 | 0.006398 | 0.134318 | 0.913793 |
| CM3216_16 | 0.152474 | −0.18198 | −0.34771 | 0.866689 | −0.37468 | −0.00266 | 3.120063 |
| CM3807_04 | 0.294057 | 1.469689 | 1.569577 | 4.761968 | 0.083238 | 0.647331 | 2.055446 |
| CM3841_02 | 0.300547 | 0.686909 | 0.165226 | −0.5379 | 0.61958 | −0.3819 | 0.309798 |
| CM4989_33 | −0.30786 | −0.24871 | 1.574781 | 0.196602 | 2.720691 | 1.944892 | 0.426132 |
| CM5300_74 | 0.057261 | 0.424526 | 0.248053 | 0.021913 | 1.277669 | −0.11381 | 0.042662 |
| CM5341_77 | −0.03085 | −0.0418 | 0.193441 | 1.688618 | 0.32415 | 0.020686 | 0.07763 |
| CM5502_92 | 0.47105 | 0.26178 | 2.213614 | 2.327421 | 0.446049 | −0.16163 | −0.85773 |
| CM6370_74 | 0.068094 | 0.212024 | 0.045106 | 0.249062 | 0.084185 | 0.56917 | 0.268651 |
| CM7157_07 | 0.008713 | 0.162608 | −0.00353 | −0.03179 | −0.00199 | 0.057586 | 0.345295 |
| CM7970_18 | 0.421992 | 0.480456 | 0.414526 | 0.128169 | 0.287635 | 0.082395 | 0.507187 |
| IM14251_6 | −0.00154 | 0.171248 | 0.175851 | 0.098494 | 0.134754 | 0.345064 | 0.203886 |
| IM19417_7 | 0.079362 | 0.082223 | 0.554148 | 0.201174 | −0.05405 | 0.991607 | 0.377345 |
| IM19700_3 | 0.006215 | 0.025034 | 0.10762 | 0.103783 | 0.405736 | 0.415707 | 0.010538 |
| IM2256_18 | −0.25038 | −0.43749 | −0.26386 | 0.623225 | 3.461164 | −0.06599 | 2.601847 |
| IM28120_5 | −0.01223 | 0.041766 | 0.432216 | 0.18821 | 0.639716 | 0.210524 | 0.433941 |
| IM28759_8 | 0.285447 | 0.127942 | 1.086442 | 0.116994 | 1.004997 | 0.567928 | 0.337487 |
| IM29582_4 | 0.187785 | 0.41167 | 0.659343 | 0.061561 | 1.730289 | 0.496646 | 1.079102 |
| IM29738_7 | 0.134474 | 0.362305 | 0.803769 | 0.218196 | 1.818929 | 0.631478 | 0.814843 |
| IM30775_5 | 0.091652 | 0.076286 | 0.105335 | 0.05596 | 1.463259 | 0.268002 | 0.621778 |
| IM31568_8 | 0.030672 | 0.191896 | 0.405252 | 0.201842 | 0.491104 | 1.493508 | 0.378703 |
| IM4022_87 | 0.651942 | 1.975959 | 6.278744 | 0.249456 | 1.752584 | 1.547295 | 2.664368 |
| IM4175_52 | 0.587978 | 1.09109 | 0.806835 | 1.651465 | 1.569314 | 1.316232 | 0.360162 |
| IM4419_59 | −0.27612 | 1.125761 | 1.038983 | 2.713758 | 4.306741 | 0.607846 | 2.761359 |
| IM4828_68 | 1.174093 | 2.40525 | 6.550326 | 2.374229 | 6.203701 | 3.706891 | 1.552409 |
| peak | N31 | N11 | N7 | N20 | N4 | N3 | N2 |
| CM10542_6 | 0.940121 | 0.221344 | 0.275687 | 0.977586 | 0.320159 | 0.147092 | 0.725317 |
| CM13930_4 | 0.148338 | 0.171006 | 0.036538 | 0.278341 | 0.278222 | 0.054256 | 0.117435 |
| CM17991_0 | 0.757577 | 0.79616 | −0.01537 | 0.502065 | 0.389225 | 0.07521 | 0.364753 |
| CM3084_16 | −0.0213 | 0.809433 | −0.10531 | −0.13073 | 0.856869 | 0.014544 | 0.003787 |
| CM3216_16 | 4.680926 | 0.760642 | 0.11947 | 0.224714 | 0.692905 | 1.274058 | −0.07955 |
| CM3807_04 | 5.065733 | 4.188078 | 2.301019 | 0.337485 | 2.054572 | 1.177572 | 1.821716 |
| CM3841_02 | −0.01515 | −0.50761 | −0.44221 | 0.25905 | −0.12715 | −0.00622 | −0.13235 |
| CM4989_33 | 1.387684 | 1.980891 | 0.937523 | 1.690785 | 0.51552 | 0.398474 | 0.635318 |
| CM5300_74 | 0.805242 | 0.125163 | −0.09875 | 2.909522 | 0.60391 | 0.963176 | 1.269394 |
| CM5341_77 | 0.498089 | 0.681764 | 0.555135 | 0.214948 | −0.73539 | 0.150361 | −0.11566 |
| CM5502_92 | −0.31371 | −0.52391 | 0.536642 | −0.22719 | 0.506064 | −0.10871 | −0.27881 |
| CM6370_74 | −0.0674 | 0.08291 | 0.432355 | 0.135934 | −0.1541 | 0.051653 | 0.799223 |
| CM7157_07 | 0.960503 | 0.280608 | 0.842371 | 0.127182 | 0.245406 | 0.13153 | 0.016645 |
| CM7970_18 | 0.339715 | 0.823071 | 0.375998 | 0.579585 | 0.513653 | 0.144706 | −0.0053 |
| IM14251_6 | 0.698632 | 0.950001 | −0.09151 | 0.576307 | 0.009453 | −0.05644 | 0.950208 |
| IM19417_7 | 0.856132 | 0.450378 | 0.085153 | 0.004042 | 0.071719 | 0.271278 | 0.149572 |
| IM19700_3 | 0.478265 | 0.269237 | 0.107788 | 0.324448 | 0.186351 | 0.166146 | 0.157516 |
| IM2256_18 | −0.29801 | 1.725645 | −0.19535 | 1.596711 | 0.728795 | 0.768844 | 0.831336 |
| IM28120_5 | 0.063646 | 0.270529 | 0.494496 | 0.554032 | 0.628011 | 0.396193 | 0.155522 |
| IM28759_8 | 0.182764 | 1.159265 | 0.56441 | 0.694227 | 0.578621 | 0.004081 | −0.01314 |
| IM29582_4 | 0.493512 | 0.852713 | 0.577745 | 0.856558 | 0.511234 | 0.142129 | 0.100595 |
| IM29738_7 | 0.709684 | 0.436591 | 0.285927 | 1.120563 | 0.534256 | 0.128891 | 0.068083 |
| IM30775_5 | 0.318032 | 0.653983 | 0.125575 | 0.262006 | 0.177468 | 0.225655 | 0.135528 |
| IM31568_8 | 0.568544 | 1.607771 | 0.169296 | 1.640406 | 0.838473 | 0.300111 | 0.076901 |
| IM4022_87 | 2.872295 | 2.95445 | 0.288891 | 2.350513 | 1.562764 | 6.425221 | 1.221158 |
| IM4175_52 | 2.086456 | 1.841332 | 0.769795 | 3.528311 | 0.48742 | 1.470774 | 1.179191 |
| IM4419_59 | 2.175627 | 0.648302 | −0.16651 | 2.70962 | 2.518304 | −0.93053 | 0.527005 |
| IM4828_68 | 5.22745 | 4.823589 | 1.749467 | 6.302119 | 3.060848 | 2.299948 | 3.077726 |

Raw dara for FIG> 1 (control samples)

| peak | C18 | C12 | C14 | C24 | C17 | C6 | C10 |
|---|---|---|---|---|---|---|---|
| CM10542_6 | 0.423127 | 0.099816 | 0.279256 | 0.199698 | 0.001213 | 0.200273 | 0.042724 |
| CM13930_4 | 0.049771 | 0.050608 | 0.151065 | −0.0027 | 0.003694 | 0.157111 | 0.071407 |
| CM17991_0 | −0.04478 | 0.386745 | 0.02983 | 0.397974 | 0.002603 | 0.136415 | 0.05017 |
| CM3084_16 | −1.23212 | −0.30002 | 0.188648 | −0.50541 | −0.0043 | −0.18695 | 0.050137 |
| CM3216_16 | 12.71117 | 2.544614 | −0.03009 | 0.517461 | −0.12575 | 1.116719 | 9.311762 |
| CM3807_04 | 2.668269 | 0.84867 | 0.353773 | −0.4488 | 0.783197 | 3.496368 | −0.21427 |
| CM3841_02 | 1.742271 | 0.204634 | −0.46312 | 0.189331 | −0.35752 | 0.244311 | 0.087019 |
| CM4989_33 | 0.355943 | 0.499513 | 1.367935 | 3.106332 | −0.00576 | 0.770478 | 4.728728 |
| CM5300_74 | 3.336231 | 0.461271 | −0.0317 | −0.27837 | −0.05256 | 6.287488 | 13.89865 |
| CM5341_77 | 4.147783 | 0.05694 | 0.214349 | −0.03501 | −0.12461 | 0.077928 | 3.35954 |
| CM5502_92 | 7.249805 | −0.64213 | 0.885744 | 6.249688 | 1.136043 | 1.602358 | 2.883372 |
| CM6370_74 | 1.073516 | 0.919055 | 0.640674 | 0.633554 | 1.31802 | 0.453068 | 0.090104 |
| CM7157_07 | 0.276932 | −0.27471 | 0.206196 | −0.06005 | 0.02429 | −0.13764 | −0.10788 |
| CM7970_18 | 0.165903 | −0.04494 | 0.083321 | 0.13312 | 0.195443 | 0.448561 | 0.098181 |
| IM14251_6 | 1.480072 | 0.373705 | 0.708147 | 0.480782 | 0.003837 | 0.28771 | 1.753913 |
| IM19417_7 | 0.339428 | 1.069743 | 0.950092 | 0.629352 | 0.085572 | 0.020428 | 0.336161 |
| IM19700_3 | 0.347921 | 0.227744 | 0.613725 | 0.628733 | 0.054897 | 0.276594 | −0.02 |
| IM2256_18 | 2.661745 | 1.672753 | 0.876073 | 0.400503 | 1.442941 | 3.437729 | 0.261096 |
| IM28120_5 | 0.683495 | 0.885178 | 0.321517 | −0.05099 | 0.086262 | 0.51815 | 0.777059 |
| IM28759_8 | 0.98018 | 1.400042 | 0.938596 | 0.304264 | 0.034038 | 0.387335 | 1.036938 |
| IM29582_4 | 0.706957 | 1.157823 | 0.303482 | 0.700271 | 0.042528 | 1.464012 | 1.434139 |
| IM29738_7 | 0.587474 | 1.533131 | 0.639911 | 0.314433 | 0.180645 | 1.534196 | 1.226322 |
| IM30775_5 | 0.642505 | 0.096894 | 0.873641 | 0.439707 | 0.02751 | 0.266257 | 0.745999 |
| IM31568_8 | 1.761968 | 1.416373 | 0.916708 | 0.819823 | 0.175281 | 0.349013 | 1.356762 |
| IM4022_87 | 1.509505 | 1.085225 | 0.119615 | −0.28464 | −0.32136 | 1.334158 | 1.073732 |
| IM4175_52 | 1.263138 | 1.678394 | 3.952243 | 3.369464 | 4.877147 | 2.217585 | 1.404272 |
| IM4419_59 | 2.319654 | 0.5157 | 1.257194 | 0.304749 | 6.791693 | 2.344135 | 2.390607 |
| IM4828_68 | 13.61952 | 1.295898 | 4.086566 | 4.930621 | 0.151364 | 3.363565 | 12.13014 |
| peak | C31 | C11 | C7 | C20 | C4 | C3 | C2 |
| CM10542_6 | 0.292349 | 0.031812 | −0.0009 | 0.349641 | 0.252389 | −0.00394 | 0.135122 |
| CM13930_4 | 0.087689 | −0.03433 | 0.050765 | 0.061274 | 0.049724 | 0.020145 | −0.02381 |
| CM17991_0 | 0.012523 | −0.01806 | −0.00343 | 0.664514 | 0.127107 | 0.022995 | −0.00455 |
| CM3084_16 | 1.060313 | 0.347595 | −0.43267 | −1.23496 | −0.1123 | −0.20096 | 0.047878 |
| CM3216_16 | 1.475516 | 10.23575 | 19.95506 | 0.963997 | 5.045665 | 0.391769 | 0.113753 |
| CM3807_04 | 1.348987 | −0.01796 | 0.312385 | −0.24594 | 1.470363 | 0.071223 | −0.32391 |
| CM3841_02 | 1.033631 | 2.47022 | 0.781511 | 0.503417 | 0.290496 | −0.02102 | 2.771945 |
| CM4989_33 | 8.815282 | 1.88258 | 1.430933 | 3.066782 | 1.266511 | 5.664059 | 3.131751 |
| CM5300_74 | 4.810181 | 1.575743 | 0.368356 | 1.022958 | 0.917644 | 1.64379 | 10.71435 |
| CM5341_77 | 0.466067 | 3.035856 | 0.546086 | 1.903203 | 4.9272 | −0.08323 | 0.585698 |
| CM5502_92 | 1.831665 | 3.173436 | 0.828577 | −0.134 | 1.161747 | 1.522434 | 17.54152 |
| CM6370_74 | 0.020515 | 0.371414 | 0.051964 | 0.164173 | 0.383168 | 0.128715 | 0.298226 |
| CM7157_07 | −0.02575 | 0.056747 | 0.097616 | −0.0145 | 0.102308 | 0.083117 | 0.050183 |
| CM7970_18 | −0.00703 | −0.08096 | 0.319959 | 0.299786 | 0.15409 | 0.182531 | −0.18285 |
| IM14251_6 | 0.797374 | 0.574881 | 1.760395 | 0.146657 | 0.424522 | 0.849297 | 0.060664 |
| IM19417_7 | 1.489702 | 0.772323 | 0.738185 | 0.453577 | 0.609166 | 1.822257 | 0.019594 |
| IM19700_3 | 0.716934 | 0.130931 | 0.395469 | 0.956042 | 0.606039 | 1.263601 | 0.024424 |
| IM2256_18 | 5.236986 | 1.252633 | 2.468293 | 1.97162 | 0.742794 | 0.386727 | 3.672978 |
| IM28120_5 | 1.613022 | 0.750945 | 2.184036 | 0.540219 | 0.789668 | 1.808027 | −0.01788 |
| IM28759_8 | 3.022978 | 0.641574 | 1.131493 | 2.13614 | 2.325956 | 2.068064 | −0.00884 |
| IM29582_4 | 0.978813 | 1.27955 | 1.152159 | 3.970599 | 1.697722 | 1.508362 | 0.138246 |
| IM29738_7 | 2.925023 | 1.06973 | 0.482887 | 3.209492 | 1.869727 | 1.452691 | 0.168935 |
| IM30775_5 | 0.923621 | 0.377048 | 1.14889 | 1.362716 | 0.493846 | 1.672685 | 0.43427 |
| IM31568_8 | 2.608251 | 1.134886 | 2.093685 | 0.646605 | 0.57323 | 3.244205 | 1.920647 |
| IM4022_87 | 0.290376 | 1.623873 | 0.575472 | 1.033566 | 1.695327 | 0.661247 | 1.000204 |
| IM4175_52 | 5.199796 | 1.35501 | 1.959846 | 2.329253 | 0.942701 | 1.096446 | 7.406805 |
| IM4419_59 | 4.991518 | 4.25435 | 4.548453 | 4.144426 | 2.526795 | 0.312384 | 4.746393 |
| IM4828_68 | 4.335978 | 2.655541 | 6.665391 | 11.93288 | 14.85669 | 7.615298 | 3.61567 |

TABLE 5
Average of Data from Table 4

| | Avg Intensity | Avg Intensity | Increased or |
|---|---|---|---|
| | Value for | Value for | Decreased |
| Biomarker | Cases | Controls | in Cases? |
| CM10542 | 0.407776 | 0.16447 | Increased |
| CM13930 | 0.13409 | 0.049458 | Increased |
| CM17991 | 0.35646 | 0.125718 | Increased |
| CM3084 | 0.24087 | −0.17965 | Increased |
| CM3216 | 0.778954 | 4.587671 | Decreased |
| CM3807 | 1.987677 | 0.721597 | Increased |
| CM3841 | 0.013616 | 0.676938 | Decreased |
| CM4989 | 0.98948 | 2.577219 | Decreased |
| CM5300 | 0.609709 | 3.191002 | Decreased |
| CM5341 | 0.248652 | 1.3627 | Decreased |
| CM5502 | 0.306495 | 3.235019 | Decreased |
| CM6370 | 0.198348 | 0.467583 | Decreased |
| CM7157 | 0.224367 | 0.019776 | Increased |
| CM7970 | 0.363842 | 0.12608 | Increased |
| IM14251 | 0.297458 | 0.692997 | Decreased |
| IM19417 | 0.294292 | 0.666827 | Decreased |
| IM19700 | 0.197456 | 0.444504 | Decreased |
| IM2256 | 0.773321 | 1.891777 | Decreased |
| IM28120 | 0.321184 | 0.777765 | Decreased |
| IM28759 | 0.47839 | 1.17134 | Decreased |
| IM29582 | 0.58292 | 1.181047 | Decreased |
| IM29738 | 0.576285 | 1.228186 | Decreased |
| IM30775 | 0.32718 | 0.678971 | Decreased |
| IM31568 | 0.599606 | 1.358388 | Decreased |
| IM4022 | 2.342546 | 0.814021 | Increased |
| IM4175 | 1.339025 | 2.789436 | Decreased |
| IM4419 | 1.411439 | 2.960575 | Decreased |
| IM4828 | 3.607718 | 6.518223 | Decreased |

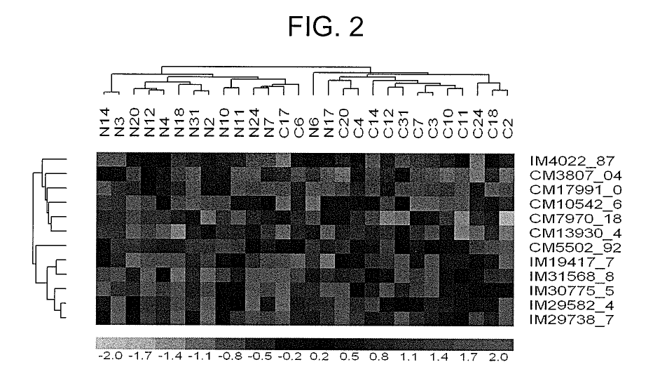

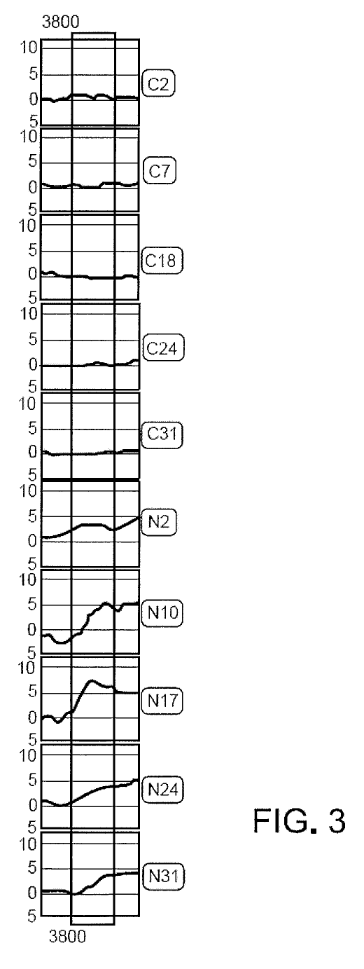

These 28 peaks were further refined into a 12-peak predictive signature based on their prediction accuracy on the training set. This 12-peak signature displayed 93% sensitivity, 86% specificity, and 89% leave-one-out cross validation accuracy (25/28 predicted accurately, P<0.001) for the development of DN. Normalizing the protein signature results for urine creatinine concentrations slightly improved the accuracy to 93%. Hierarchical clustering of the samples in the training set using the 12-peak signature is shown in FIG. 2. Control samples are denoted by C, and case samples by N. In FIG. 3, tracings from the SELDI-TOF spectra for one representative peak (CM3807_{—}04) in 10 samples from the training group (5C, 5N) are highlighted. As suggested by FIG. 1, this peak is elevated in case and not control samples.

When this 12-peak signature was applied on the validation set, the overall accuracy was 74% (25/34 predicted correctly; 5 case and 4 controls predicted incorrectly, p=0.01), with a sensitivity of 71%, and a specificity of 76%.

The intensity values for the peaks in the 12-peak signature set can be found in bold in the data in Table 4. Average intensity values are shown in Table 5, along with an indication of whether an increase or a decrease in a biomarker is associated with a propensity to develop DN.

The distribution of factors known to be associated with diabetic nephropathy between cases and controls was examined in detail. Most characteristics, including blood pressure and blood pressure medication use, were not different at baseline between the two groups. Alternatively, cases demonstrated higher HbA1c levels compared with controls (see Table 1). In a multivariate binary logistic regression model adjusting for baseline HbA1c, the SELDI 12-peak signature was independently predictive of diabetic nephropathy in the validation set (Odds Ratio, OR, 7.9, 95% CI 1.5-43.5, p=0.017), as well as in the entire dataset (OR, 14.5, 95% CI 3.7-55.6, p=0.001), and in both analyses HbA1c was no longer significantly associated with subsequent DN.

Protein biomarker profiling can be used to identify subjects at risk for developing DN about 5-10 years before the development of DN, and even before the development of microalbuminuria.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of determining whether a test subject is predisposed to develop diabetic nephropathy (DN), the method comprising:
obtaining a urine sample from the test subject;
measuring the level of at least one biomarker, the molecular weight of which is listed in Table 2, in the test subject's urine sample;
comparing the level of the at least one biomarker in the test subject's urine sample to a reference level that represents a level of the same biomarker in a reference subject's urine sample; and
determining whether the test subject is predisposed to develop DN based on the comparison of the level of the at least one biomarker in the test subject's urine sample with the level of the at least one biomarker in the reference subject's urine sample.

2. The method of claim 1, wherein the method comprises measuring the level of two or more of the biomarkers, the molecular weights of which are listed in Table 2.

3. The method of claim 1, wherein the method comprises measuring the level of all of the biomarkers, the molecular weights of which are listed in Table 2.

4. The method of claim 2, further comprising generating a subject profile comprising one or more values, each value representing a level of a biomarker; comparing the subject profile with a reference profile, wherein the reference profile comprises one or more values, each value representing a level of the same biomarkers in a reference urine sample obtained from a reference subject; and
determining whether the subject is predisposed to having DN based on the comparison of the subject profile with the reference profile.

5. The method of claim 1, wherein the reference level represents a level of the biomarker in a urine sample obtained from a subject who has a predisposition to develop DN.

6. The method of claim 1, wherein the reference level represents a level of the biomarker in a urine sample obtained from a subject who does not have a predisposition to develop DN.

7. The method of claim 1, comprising comparing the level of the at least one biomarker in the test subject's urine sample to a reference level that represents a level of the same biomarker in urine samples from both a reference subject who has a predisposition to DN and a reference subject who does not have a predisposition to develop DN.

8. The method of claim 1, wherein the level of the at least one biomarker is measured using surface-enhanced laser desorption/ionization-time of flight-mass spectrometry (SELDI-TOF-MS).

9. The method of claim 1, wherein a predisposition to develop DN is determined when the comparison indicates an increase or decrease in the subject biomarker level or levels compared to the reference biomarker levels or levels, as shown in Table 5.

10. The method of claim 1, wherein measuring the level of at least one biomarker comprises contacting the sample to an array of two or more immobilized biomarker-specific biomolecules, and detecting binding of the biomarker to the immobilized biomolecules.

11. The method of claim 10, wherein the biomarker-specific biomolecules are antibodies.

12. The method of claim 11, wherein the antibodies are monoclonal antibodies.

13. An array for detecting a disposition to diabetic nephropathy (DN), the array comprising a substrate having a plurality of addresses, each address having disposed thereon a set of one or more biomolecules, and each biomolecule in the set at a given address specifically detecting the same biomarker; wherein the array comprises sufficient addresses to detect at least two or more of the biomarkers, the molecular weights of which are listed in Table 2.

14. The array of claim 13, comprising sufficient addresses to detect all of the biomarkers, the molecular weights of which are listed in Table 2.

15. A pre-packaged diagnostic kit for detecting a predisposition to develop diabetic nephropathy, the kit comprising an array of claim 13, and instructions for using the array.

16. A method of determining whether a therapy is effective for reducing a predisposition to diabetic nephropathy in a subject, the method comprising:
obtaining a urine sample from a subject before administration of the therapy and determining a level of at least one biomarker, the molecular weight of which is listed in Table 2, in the sample to generate a pre-therapy biomarker level;
obtaining a urine sample from a subject undergoing the therapy and determining a level of the biomarker to generate a therapy biomarker level; and
comparing the pre-therapy biomarker level to the therapy biomarker level;
wherein a difference in the pre-therapy biomarker level compared to the therapy biomarker level indicates whether the therapy is effective.

17. A method of monitoring a subject's predisposition to develop diabetic nephropathy, the method comprising:
obtaining a first urine sample from a subject and determining a level of at least one biomarker, the molecular weight of which is listed in Table 2, in the sample to generate a baseline biomarker level;
obtaining a second urine sample from the subject and determining a level of the biomarker to generate a test biomarker level; and
comparing the baseline biomarker level to the test biomarker level;
wherein the comparison of the baseline biomarker level to the test biomarker level indicates whether the subject's propensity to develop DN is reduced, increased, or unchanged.

18. The method of claim 16, wherein the level of the at least one biomarker is measured using surface-enhanced laser desorption/ionization-time of flight-mass spectrometry (SELDI-TOF-MS).

19. The method of claim 17, wherein the level of the at least one biomarker is measured using surface-enhanced laser desorption/ionization-time of flight-mass spectrometry (SELDI-TOF-MS).
